(19) United States
(12) Patent Application Publication
(10) Pub. No.: US 2025/0120781 A1
(43) Pub. Date: Apr. 17, 2025

(54) MEDICAL DEVICE TRANSPORTATION CART AND OPERATION METHOD OF MEDICAL DEVICE TRANSPORTATION CART

(71) Applicant: OLYMPUS MEDICAL SYSTEMS CORP., Tokyo (JP)

(72) Inventor: Takeo SUZUKI, Tokyo (JP)

(73) Assignee: OLYMPUS MEDICAL SYSTEMS CORP., Tokyo (JP)

(21) Appl. No.: 18/910,158

(22) Filed: Oct. 9, 2024

Related U.S. Application Data
(60) Provisional application No. 63/543,741, filed on Oct. 12, 2023.

(51) Int. Cl. A61B 50/13; A61B 90/00; A61B 90/90

(52) U.S. Cl. CPC A61B 50/13; A61B 90/36; A61B 90/90

(57) ABSTRACT

A medical device transportation cart includes: a cart body; a storage area configured to store a medical device; and at least one processor. The at least one processor configured to: determine a first number of the medical device contained in the storage area at a first time; determine a second number of the medical device contained in the storage area at a second time; and when the first number and the second number are different, control an actuator to restrict generation of the propulsion force.

### CROSS REFERENCE TO RELATED APPLICATION

This application is based on and claims priority under 35 U.S.C. § 119 to U.S. Provisional Application No. 63/543,741 filed on Oct. 12, 2023, the entire contents of which are incorporated herein by reference.

### FIELD OF DISCLOSURE

The disclosure relates to a medical device transportation cart for transporting medical device and an operation method of the medical device transportation cart.

### BACKGROUND

Medical devices used for various medical procedures conducted in medical institutions are often stored in a location different from the place where the medical procedure is performed, such as an examination room (endoscope room). During an examination, the medical devices are in general brought from the storage location to the examination room, etc. For example, during an endoscopic procedure, a cart equipped with a control apparatus, a scope, etc. is transported to the endoscope room and the cart is placed in the endoscope room, to thereby carry out the endoscopic procedure.

When the endoscope used in the procedure is a single-use endoscope, a cart equipped with a control device is transported to the endoscope room, mounting thereon a plurality of scopes, etc., as many as necessary for the procedure, and an endoscope corresponding to the procedure is used for each procedure. After the medical device is used, the medical devices are collected and transported to a disposal site, etc., by a cart, etc.

Single-use endoscopes should not just be simply discarded after use, but should be subjected to appropriate treatments such as, e.g., treatment for preventing infection, treatment for reusing some of the medical device, treatment as medical waste, and treatment to prevent reuse. For this reason, it is preferable to collect all used medical devices, especially single-use medical devices, stored in a designated collection container. This requires a system that prevents a medical professional from forgetting to put used single-use medical devices in a collection section provided on a cart after the end of a procedure. The medical device can be one or more of an endoscope, a single-use endoscope, a treatment instrument and a single-use treatment instrument. A single-use endoscope is disposed of (discarded) after being used once, and should not be used multiple times. The single-use endoscope can include a single-use component and a multi-use component. The single-use component is disposed or returned to manufacturer of after being used once, and the multi-use component is a reusable component that is repeatedly used. Even the reusable endoscope capable of being used multiple times by being reprocessed can be stopped being used and be sent to the manufacturer or the like for maintenance after being used a predetermined number of times.

Note that Japanese Patent Application Laid-Open Publication No. 10-286219 (hereinafter referred to as Patent Document 1) discloses an endoscope medical device storage device that is transportable with endoscopic medical devices stored therein and that is equipped with a cooperative means (e.g., a wheel drive motor) that cooperates with the device during transportation to facilitate transportation. Japanese Patent Laid-Open Publication No. 2020-104843 (hereinafter referred to as Patent Document 2) discloses an electric handcart that obtains a gain by an output from the sensor that detects a gripping strength of the grip gripped by the operator with respect to the motor that runs the drive wheels of the endoscopic surgical apparatus, obtains an amount by which the input operation amount is amplified by the gain, to obtain the target rotation speed of the motor. Japanese Patent Laid-Open Publication No. 2004-201897 (hereinafter referred to as Patent Document 3) discloses a service cart that has four wheels and is driven by an operator applying an operating force with a handle, the service cart being configured to have assist wheels that are driven by a motor that is disposed integrally provided under the floor board and are capable of power assisting the running, and a controller that controls the driving force of the motor in response to the operating force detected by a mount sensor that detects the operating force applied to the handle.

### SUMMARY

A medical device transportation cart includes: a cart body; a storage area configured to store a medical device; and at least one processor. The at least one processor configured to: determine a first number of the medical device contained in the storage area at a first time; determine a second number of the medical device contained in the storage area at a second time; and when the first number and the second number are different, control an actuator to restrict generation of the propulsion force

An operating method of a transportation cart comprises: determining a first number of a medical device contained in the storage area at a first time; determining a second number of the medical device contained in the storage area at a second time; and when the first number and the second number are different, control an actuator to restrict generation of a propulsion force for moving the cart body.

### BRIEF DESCRIPTION OF THE DRAWINGS

FIG. 1 is a perspective view showing an example of an appearance of a medical device transportation cart according to an embodiment of the disclosure.

FIG. 2 is a perspective view showing another example of the medical device transportation cart.

FIG. 3 is a perspective view showing a hanger section 35 attached to a support pillar 35a.

FIG. 4 is a perspective view showing a collection box 50.

FIG. 5 is an explanatory diagram showing the storage of an unused endoscope 41 in an endoscope storage case 40.

FIG. 6 is an explanatory diagram for describing the installation of the collection box 50.

FIG. 7 is a perspective view showing a configuration of a caster 11 in FIGS. 1 and 2.

FIG. 8 is a block diagram showing an example of a configuration of a control apparatus 18.

FIG. 9 is a flow chart for describing an operation of the embodiment.

FIG. 10 is a perspective view showing an example of a state in which a collection-requiring medical device storage section 30 is not used in a cart C.

FIG. 11 is a perspective view showing a modification.

### DETAILED DESCRIPTION

An embodiment of the disclosure is described in detail below with reference to the drawings.

### Embodiment

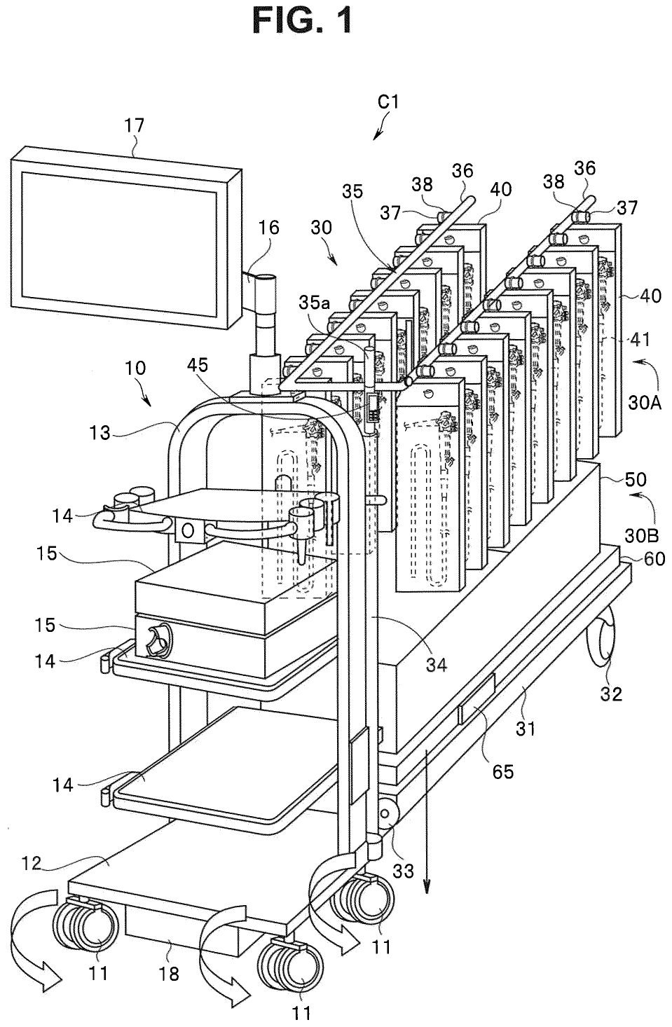

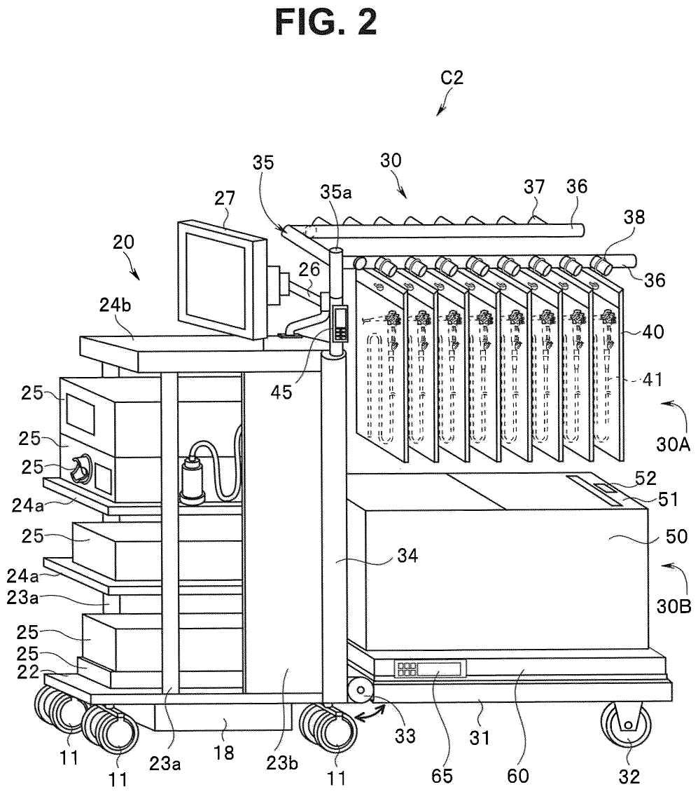

FIG. 1 is a perspective view showing an example of an appearance of a medical device transportation cart according to an embodiment of the disclosure. FIG. 2 is a perspective view showing another example of the medical device transportation cart, showing a medical device transportation cart with a different appearance from that in FIG. 1. Note that the drawings are schematic, and the dimensional ratios, etc. of each part are different from those in reality. Of course, the drawings also include parts with different dimensional relationships and ratios.

This embodiment allows to, when transporting unused and used medical devices by a medical device transportation cart, control the power assist of forward and backward movements of the medical device transportation cart based on the weight difference between the unused medical devices and the used medical devices, thereby allowing the medical professional to recognize whether the medical devices has been surely collected. In this embodiment, medical devices including an endoscope are described as an example of the medical device, but the medical devices do not have to include an endoscope, and the embodiment can be applicable to medical device transportation carts that transport various medical devices.

The above-mentioned Patent Documents 1 to 3 disclose a technology that assists (power assists) the force used by the user to move a cart used for an endoscope, making it easier to move the cart. However, Patent Documents 1 to 3 do not include the idea of assisting the movement only when all medical devices such as endoscopes have been collected, and do not disclose a technology for controlling the assistance of the forward and backward movements of the cart based on the weight difference between the medical devices before and after use.

In this embodiment, in order to perform such assist control, a function for measuring the weight of medical devices can be provided in the medical device transportation cart (hereinafter, simply referred to as “cart”).

The cart in this embodiment can be characterized by a configuration of an unused endoscope holding section, a used endoscope storage section, and an assist apparatus, and the external shape and other configurations are not limited. The unused endoscope holding section, the used endoscope storage section, and the assist apparatus are also characterized by functions thereof, that is, the function of the unused endoscope holding section as a holding section that holds unused endoscopes, can be unused medical devices, the function of the used endoscope storage section as a storage section that stores used endoscopes, can be used medical devices, and the function of the assist apparatus that assists the forward and backward movements of the cart, and the external shape, etc. are not limited.

A cart C1 in FIG. 1 includes an endoscope apparatus storage section 10, and a collection-requiring medical device storage section 30 that configures an unused endoscope holding section 30A (first storage area) and a used endoscope storage section 30B (second storage area). A cart C2 in FIG. 2 has an endoscope apparatus storage section 20 and a collection-requiring medical device storage section 30. The endoscope apparatus storage section 10 of the cart C1 and the endoscope apparatus storage section 20 of the cart C2 each configure a cart body including frames (frame base and frame side wall). The cart C1 in FIG. 1 and the cart C2 in FIG. 2 can be functionally similar to each other.

The endoscope apparatus storage section 10 of the cart C1 has a frame base 12 provided with casters 11, a U-shaped frame side wall 13 erected on the frame base 12, and a plurality of shelf boards 14 attached to the frame side wall 13. At least one of the casters 11 provided on the bottom surface of the frame base 12, e.g., at four corners, can be equipped with a motor as described below, and has a function of assisting the forward and backward movements of the cart C1 when the user moves forward and backward the cart C1.

On the upper surface of the frame base 12 and the upper surface of each of the shelf boards 14, there can be mounted a medical device 15 such as an endoscope apparatus (endoscope control apparatus). A monitor arm 16 can be attached to the upper end of the frame side wall 13, and a monitor 17 can be attached to the monitor arm 16. On the bottom surface of the frame base 12, there can be attached a control apparatus (cart control apparatus, at least one processor) 18 and a battery (not shown in FIG. 1) that supply electric power to the motor. The battery, the control apparatus 18, and the caster 11 with a motor configure an assist apparatus. Providing the control apparatus 18 and the battery on the bottom surface of the frame base 12 enables to lower the center of gravity of the cart C1.

The endoscope apparatus storage section 20 of the cart C2 has a frame base 22 provided with the casters 11, a frame pillar 23a and a frame side wall 23b that can be erected on the frame base 22, and two shelf boards 24a and a top board 24b that can be attached to the frame pillar 23a and the frame side wall 23. The casters 11 provided on, e.g., four corners of the bottom surface of the frame base 22 have a function of assisting the user to move forward and backward the cart C2 when moving forward and backward the cart C2. A medical device transportation cart can include: the cart body (10, 20); the storage area (30A, 30B) configured to store the medical device; and at least one processor 18. the storage area can comprise the first storage area 30A configured to contain the medical device before use; and the second storage area 30B configured to store the medical device after use.

A medical device 25 such as an endoscope apparatus can be mountable on the upper surface of the frame base 22 and the upper surface of the each shelf board 24a. A monitor arm 26 can be attached to the upper end of the top board 24b, and a monitor 27 can be attached to the monitor arm 26. A control apparatus (cart control apparatus, at least one processor) 18 and a battery (not shown in FIG. 2) that supply electric power to the motor can be attached on the bottom surface of the frame base 12. In other words, the cart C2 also has an assist apparatus. By providing the control apparatus 18 and the battery on the bottom surface of the frame base 22, it can be enabled to lower the center of gravity of the cart C2.

In the example in FIGS. 1 and 2, the collection-requiring medical device storage section 30 of the carts C1, C2 (hereinafter, these will be collectively referred to as cart C when need not to be distinguished) has the same configuration and includes the unused endoscope holding section 30A and the used endoscope storage section 30B. The unused endoscope holding section 30A includes a hanger section 35 for holding unused endoscopes, can be unused medical devices. The used endoscope storage section 30B includes a collection box 50 for storing used endoscopes, can be used medical devices.

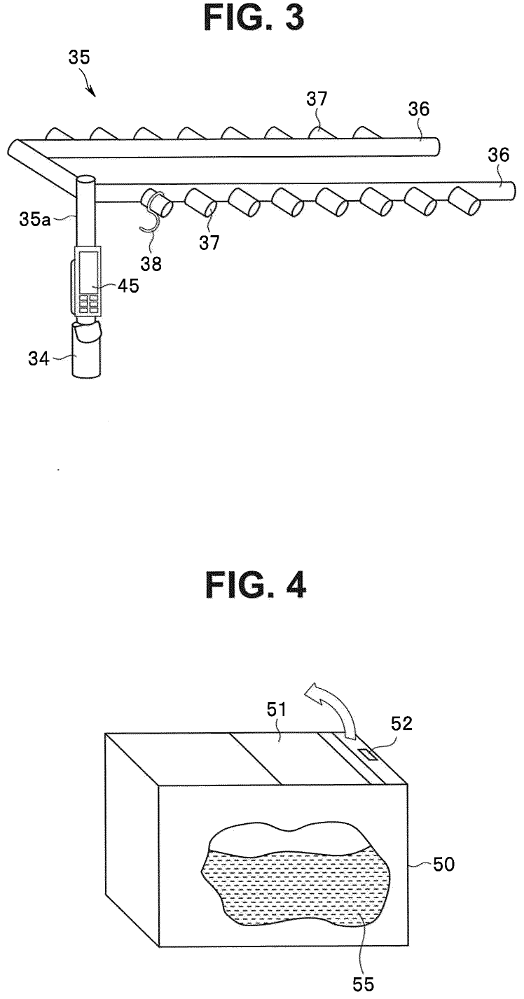

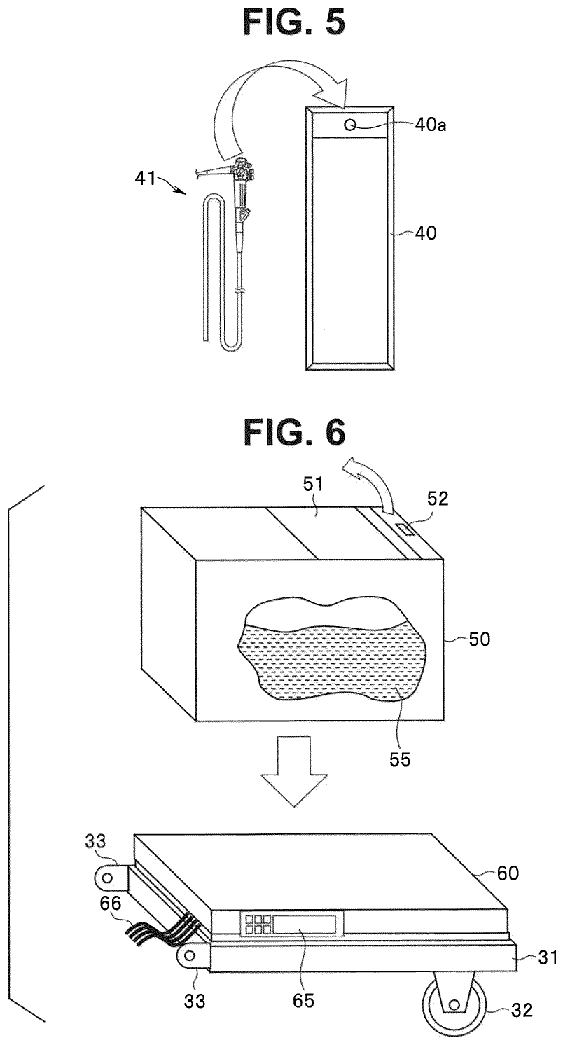

FIG. 3 is a perspective view showing a hanger section 35 attached to a support pillar 35a. FIG. 4 is a perspective view showing the collection box 50. FIG. 5 is an explanatory diagram showing the storage of an unused endoscope 41 in an endoscope storage case 40. FIG. 6 is an explanatory diagram for describing the installation of the collection box 50.

As shown in FIGS. 1 and 2, it can be configured such that a support member 34 extending in a vertical direction along the frame side wall 13 or 23b can be attached to the rear end of the frame side wall 13 or 23b, and the hanger section 35 can be attached to the support member 34.

As shown in FIG. 3, the hanger section 35 configuring the unused endoscope holding section 30A has the support pillar 35a erected at the upper end of the support member 34 and a U-shaped holding member 36 fixed to the support pillar 35a and extending in a horizontal direction. The holding member 36 can be provided with a plurality of locking sections 37 protruding from the holding member 36 at a plurality of positions at predetermined intervals in the longitudinal direction of the holding member 36. A hook 38 can be locked to the locking sections 37. It can be configured such that, by hooking the hook 38 into a locking hole 40a (FIG. 5) of the endoscope storage case 40, the endoscope storage case 40 can be hung from the locking sections 37. The endoscope storage case 40 can be, e.g., a sterilized pack that stores the unused endoscope 41, such as, e.g., a single-use endoscope, can be an unused medical device, as shown in FIG. 5. In other words, the unused endoscope 41 can be stored in the endoscope storage case 40, can be a package whose inside can be kept in a sterile state. In this way, a plurality of unused endoscopes 41 can be held to the hanger section 35, in a state stored in the endoscope storage case 40. After a single use, the single-use endoscope can be stored in the collection box 50, will be described later, and subsequently disposed of.

In this embodiment, a weight measurement unit 45 can be provided on the support pillar 35a of the hanger section 35. That is, the weight measurement unit 45 can be placed at a portion that supports the weight of the hanger section 35 that configures the unused endoscope holding section 30A. The weight measurement unit 45 measures the sum of the weight of the hanger section 35 and the weight of the endoscope storage case 40 in which the unused endoscope 41 can be stored. The weight information (measurement result) measured by the weight measurement unit 45 can be supplied to the control apparatus 18 via a signal line not shown.

The used endoscope storage section 30B can be placed on a wheeled base 31 connected to the frame base 12 or 22. The wheeled base 31, which functions as a frame supporting the used endoscope storage section 30B, has a caster 32 attached to each of, e.g., two locations on the rear end of the bottom surface, and can be connected to the frame base 12 or 22 at a connecting section 33. The connecting section 33 of the wheeled base 31 can be supported by a rotation shaft provided at the rear end of the frame base 12 and the frame base 22. As shown in FIG. 6, on the wheeled base 31 can be placed an installation and measurement table 60 serving as a scale configuring a weight measurement unit 65, can be a second weight measurement unit, and the collection box 50 can be placed on the installation and measurement table 60. Note that when the collection box 50 can be not installed on the wheeled base 31, the wheeled base 31 can be able to rotate around the connecting section 33 and fold to the rear end of the cart C1, C2.

As shown in FIG. 4, the collection box 50 for storing used medical devices has, e.g., a box shape, that allows pouring-in of a disinfectant, and can be liquid-tight against the outside, and can be capable of storing used medical devices such as single-use endoscopes used in the procedure. In the example in FIG. 4, the collection box 50 can be filled with a disinfectant 55 that allows performing simple disinfection. The collection box 50 may also be provided with a reading section 52 (light sensor, image sensor, magnetic sensor) that reads information such as image information, model information, unique ID information, and various codes of the medical device such as an endoscope to be stored, when a lid 51 of the collection box 50 can be opened. The reading section 52 (light sensor, image sensor, magnetic sensor) can be provided at one or more of the cart body, the frame, the first storage section 30A, or the second storage section 30B.

The reading section 52 can be able to employ a near-field wireless receiving apparatus such as an RFID, a reader or camera for barcodes or two-dimensional codes, etc. For example, a two-dimensional code indicating information on the medical device to be stored in the endoscope storage case 40 may be attached to the endoscope storage case 40. The information read by the reading section 52 allows to determine the type and weight of the medical device. By using the information from the reading section 52, a control section 81 described later may count a quantity, determine whether a correct model has been stored in the collection box 50, determine the weight and individual identification for each model, and perform control to permit the assist apparatus to generate a second propulsion force.

The weight measurement unit 45 may be provided with a switch for turning on the power of the weight measurement unit 45 and keys for inputting information on the medical device to hold, and may also be provided with a display for displaying a measurement result, etc. For example, the keys on the weight measurement unit 45 may be designed to be used to input information on the number of unused medical devices, information on the weight of the endoscope storage case 40, etc.

In this embodiment, the weight measurement unit 65 including the installation and measurement table 60 can be designed to measure the weight of the collection box 50 in which used medical devices can be stored. That is, the weight measurement unit 65 can be placed at a part that supports the weight of the collection box 50 that configures the used endoscope storage section 30B. The weight information (measurement result) measured by the weight measurement unit 65 can be supplied to the control apparatus 18 via a signal line 66.

The weight measurement unit 65 may be provided with a switch for turning on the power of the weight measurement unit 65 and keys for inputting information on the medical device to hold, and may also be provided with a display for displaying a measurement result, etc. For example, the keys on the weight measurement unit 65 may be designed to be used to input, e.g., weight information of the collection box 50 and the disinfectant 55.

FIGS. 1 and 2 each described an example in which the collection box 50 can be mounted on the wheeled base 31, but the collection box 50 may be mounted on the endoscope apparatus storage section 10 or the endoscope apparatus storage section 20 of the cart C as long as having a function for measuring the weight of the collection box 50. The use of the wheeled base 31 has the advantage of ensuring sufficient mounting space and lowering the height of the center of gravity of the cart C.

In the examples in FIGS. 1 and 2, the hanger section 35 configuring the unused endoscope holding part 30A can be provided above the wheeled base 31 configuring the used endoscope storage part 30B, thus effectively utilizing the installation space.

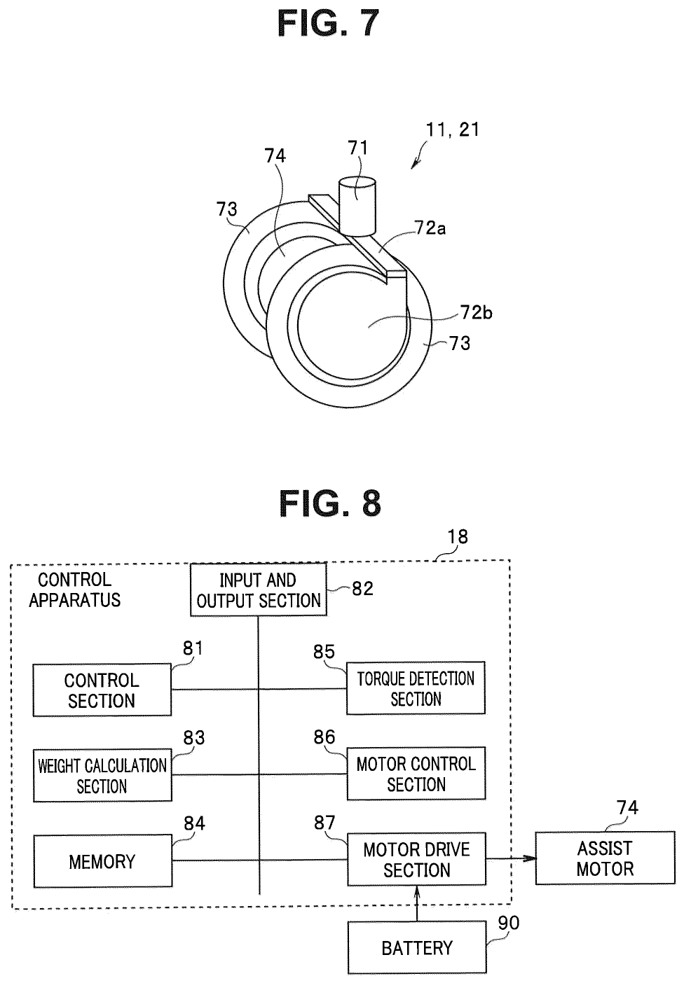

FIG. 7 is a perspective view showing a configuration of the caster 11 in FIGS. 1 and 2.

The caster 11 can be provided with, e.g., an assist motor 74 (actuator) for a function of an assist apparatus for propelling the cart, the function supplementing a first propulsion force applied by the user to generate a second propulsion force. The caster 11 can be attached to the frame base 12, 22 by a mounting section 71. The mounting section 71 can be provided with a mounting seat 72a. A bracket 72b can be attached to the mounting seat 72a, and a pair of wheels 73 can be rotatably attached to a shaft (not shown) supported by the bracket 72b. The assist motor 74 can be provided between the pair of wheels 73. The assist motor 74 can be configured such that the rotational force of the rotation shaft thereof assists the rotation of the wheel 73. A caster may be employed having one assist motor 74 per one wheel.

The assist motor 74 can be driven and controlled by the control apparatus 18. The caster 11 can be also provided with a torque sensor (not shown) that detects the rotational torque of the wheel 73 and outputs a detection signal. The torque sensor can be for detecting the first propulsion force generated by the user pushing and pulling the cart C. The at least one processor can be configured to: receive a signal from a detector of application to the cart body of a second propulsion force for moving the cart body, the second propulsion force is applied from a medical person, and in response to receiving the signal, control the actuator to generate the first propulsion force.

FIG. 8 is a block diagram showing an example of a configuration of the control apparatus 18.

The control apparatus 18 includes the control section 81, an input and output section 82, a weight calculation section 83, a memory 84, a torque detection section 85, a motor control section 86, and a motor drive section 87. The control section 81 collectively controls the entirety of the control apparatus 18. Each of the control section 81 and the control apparatus 18 may be configured by a processor using a CPU (Central Processing Section), etc., may operate according to a program stored in a memory (not shown) to control the each, or may implement some or all of the functions by a hardware electronic circuit.

The input and output section 82 can be capable of receiving a signal from the outside and outputting a signal from the control section 81 to the outside. For example, the input and output section 82 may receive an operation signal and various data based on a user operation on an operation section (not shown) and provide the reception to the control section 81. Also, for example, the input and output section 82 may output display data and an audio signal that can be generated by the control section 81 to monitor 17, 27, etc. In this embodiment, the input and output section 82 receives measurement results of the weight measurement units 45, 65 and a detection signal from the torque sensor of the caster 11 and outputs the reception to the control section 81.

The control section 81 provides the received measurement result of the weight of the unused endoscopes, i.e., the total weight of the unused medical devices, and the measurement result of the weight of the used endoscopes, etc., i.e., the total weight of the used medical devices, to the memory 84 for storage (registration). The measurement result of the weight measurement unit 45 includes the weights of the hanger section 35 and the endoscope storage case 40. The measurement result of the weight measurement unit 65 includes the weights of the collection box 50 and the disinfectant 55. In this embodiment, the power assist of the assist motor 74 can be controlled based on the difference between the total weight of the unused medical devices and the total weight of the used medical devices. As long as the difference between the total weight of the unused medical devices and the total weight of the used medical devices can be known, there is no problem even if the output of the weight measurement units 45, 65 includes the weight of items other than the medical devices.

For example, the weight of the hanger section 35 and the weight of the endoscope storage case 40 may be recorded in the memory 84, and the weight calculation section 83 may subtract these weights from the weight of the measurement result of the weight measurement unit 45, to obtain the weight of the unused medical devices and record the obtained weight in the memory 84. The weight of the collection box 50 and the weight of the disinfectant 55 may be recorded in the memory 84, and the weight calculation section 83 may subtract these weights from the weight of the measurement result of the weight measurement unit 65, to obtain the weight of the used medical devices and record the obtained weight in the memory 84. It may also be designed such that the weight of members other than the unused medical devices and the used medical devices may be input in advance by the key input of the weight measurement units 45, 65, so that the weight obtained by subtracting these weights can be measured by the weight measurement units 45, 65. It may also be designed such that the information by key input of the weight measurement units 45, 65 may be provided to the weight calculation section 83, and the weight calculation section 83 performs calculation of subtracting in advance the weight of the members other than the unused medical devices and the used medical devices. If the weight of the endoscope storage case 40 can be sufficiently smaller than the weight of the unused medical devices, the weight of the endoscope storage case 40 may be ignored.

It may also be designed such that the reading section 52 extracts information on the weight of the endoscope storage case 40 that stores the unused medical device, and the weight calculation section 83 obtains the weight obtained by subtracting the weight of the endoscope storage case 40 from the weight of the collection box 50.

For the sake of simplicity, the following description assumes that the measurement result of the weight measurement unit 45 indicates the total weight of the unused medical devices held by the hanger section 35, that the measurement result of the weight measurement unit 65 indicates the total weight of the used medical devices stored in the collection box 50, and that the information on these total weights can be registered in the memory 84.

Also stored in the memory 84 can be information on the weight of one medical device, e.g., the weight of one unused endoscope. The weight calculation section 83 can be controlled by the control section 81 to obtain the difference between the weight of the unused medical devices and the weight of the used medical devices, the weights being stored in the memory 84, and obtain whether the difference can be equal to or greater than the weight of one unused medical device. That is, the weight calculation section 83 obtains whether the number of unused medical devices held by the unused endoscope holding section 30A can be the same as the number of used medical devices stored in the used endoscope storage section 30B.

The torque detection section 85 accepts a detection signal of the torque sensor received by the input and output section 82, detects the rotational torque of the wheel 73, and outputs the detection result to the control section 81. The motor drive section 87 uses electric power from the battery 90 to drive and rotate the assist motor 74 of the caster 11. The motor drive section 87 can be controlled by the motor control section 86 to drive and rotate the assist motor 74. The motor control section 86 can be controlled by the control section 81 to control the motor drive section 87 so as to obtain a predetermined assist force (second propulsion force) according to the detection result of the rotational torque (detection result of the first propulsion force). That is, when the control section 81 detects, based on the detection result of the torque detection section 85, that the cart C has been moved forward and backward by the medical professional, the control section 81 controls the motor control section 86 so that the motor drive section 87 drives the assist motor 74 to assist the forward and backward movements of the cart C.

In this embodiment, the control section 81 can be designed to detect whether or not the medical devices transported by the cart C have all been stored in the collection box 50 as used medical devices, after use, and if all have been stored, permit the assist apparatus to generate the second propulsion force. In this embodiment, the control section 81 detects the storage state of the collection box 50 based on the calculation result of the weight calculation section 83. That is, if the calculation result of the weight calculation section 83 indicates that the difference between the weight of the unused medical devices and the weight of the used medical devices mounted on the cart C can be less than the weight of one unused medical device (i.e., the both numbers can be the same), the control section 81 determines that all of the used medical devices have been collected in the collection box 50 (hereinafter referred to as “all collected”), and generates the second propulsion force to supplement the first propulsion force applied by the user. If the difference between the weight of the unused medical devices mounted on the cart C and the weight of the used medical devices can be equal to or greater than the weight of one unused medical device, the control section 81 determines that the number of used medical devices stored in the used endoscope storage section 30B can be smaller than the unused medical devices held in the unused endoscope holding section 30A, and that at least a part of the used medical devices has not been collected in the collection box 50 (hereinafter referred to as “not all collected”), and suppresses the generation of the second propulsion force. For example, in the case of “not all collected”, the control section 81 does not generate the second propulsion force or generates a third propulsion force weaker than the second propulsion force. In the case of “not all collected”, the control section 81 may perform control on the assist apparatus to fix its operation, i.e., control to lock control the wheels 73. The at least one processor 18 can be configured to determine a first total number of medical devices 41, wherein the first total number of medical devices 41 can be a sum of a number of medical devices 41 contained in the first storage area 30A determined at a first time and a number of medical devices 41 contained in the second storage area 30B determined at the first time, and determine a second total number of medical devices 41, wherein the second total number of medical devices 41 can be a sum of a number of medical devices 41 contained in the first storage area 30A determined at a second time and a number of medical devices contained in the second storage area 30B determined at the second time. Determining whether the first and second storage areas 30A, 30B contains the medical device can comprise determine a difference in number between the first total number and the second total number. The at least one processor 18 can be further configured to, when the first total number and the second total number can be different, control the actuator 74 to prevent generation of the propulsion force. The at least one processor 18 can be configured to, when the second total number can be smaller than the first total number, control the actuator 74 to restrict generation of the propulsion force. The at least one processor 18 can be configured to, when the first number and the second number are different, prevent the actuator 74 not to generate the propulsion force. The at least one processor 18 can be configured to, when the second number is smaller than the first number, control the actuator 74 not to generate the propulsion force.

### (Operation)

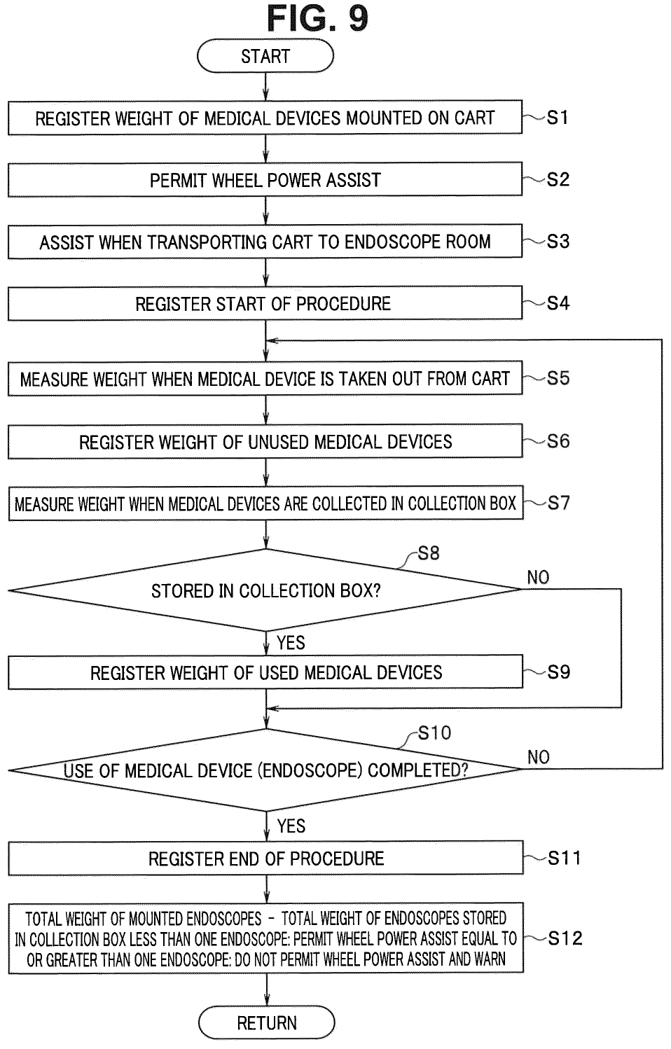

Next, an operation of the embodiment thus configured will be described with reference to FIG. 9. FIG. 9 is a flowchart for describing an operation of the embodiment.

When performing an endoscopic procedure, the medical professional transports endoscopes required for the procedure from the storage to the examination room, i.e., the endoscope room. That is, the medical professional moves the cart C to the storage, mounts the endoscopes on the cart C by a number required for the endoscopic procedure, and mounts the collection box 50 on the cart C. Note that the medical professional mounts all the endoscopes on the cart C by hanging the endoscope storage case 40 on the hanger section 35 with the endoscopes stored one by one in the endoscope storage case 40. The at least one processor 18 can be configured to determine a first number of the medical device 41 contained in the storage area (30A, 30B) at a first time; determine a second number of the medical device 41 contained in the storage area (30A, 30B) at a second time; and when the first number and the second number are different, control the actuator 74 to restrict generation of the propulsion force. The first number of the medical device 41 is a number of the medical device 41 contained the first and the second storage areas (30A, 30B) at the first time, and the second number of the medical device 41 is a number of the medical device 41 contained the first and the second storage areas (30A, 30B) at the second time. The at least one processor 18 can be configured to: determine whether the first storage area 30A contains the medical device 41; determine whether the second storage area 30B contains the medical device 41; and when one of the first storage area 30A and the second storage area 30B can be determined to contain the medical device 41, control the actuator 74 to allow generation of the propulsion force for moving the cart body. The at least one processor 18 can be configured to: when both the first and the second storage areas (30A, 30B) can be determined to not contain the medical device 41, control the actuator 74 to prevent generation of the propulsion force. The propulsion force can be a first propulsion force, and the at least one processor 18 can be configured to: when both the first and the second storage areas (30A, 30B) can be determined to not contain the medical device 41, control the actuator 74 to allow generation of a second propulsion force for moving the cart body. The second propulsion force can be less than the first propulsion force. The transportation cart can comprise: the cart body; the storage area 30A, 30B configured to store the medical device 41; the actuator 74 configured to generate the propulsion force for moving the cart body; and the at least one processor 18. The at least one processor 18 can be configured to: determine a first number of the medical device 41 contained in the storage area at a first time, determine a second number of the medical device 41 contained in the storage area at a second time, when the first number and the second number are different, control the actuator 74 to restrict generation of the propulsion force, and when the first number and the second number are the same, control the actuator 74 to allow generation of the propulsion force. The operating method of the transportation cart can comprises determining the first number of the medical device 41 contained in the storage area (30A, 30B) at the first time; determining the second number of the medical device 41 contained in the storage area (30A, 30B) at the second time; and when the first number and the second number are different, control the actuator 74 to restrict generation of the propulsion force for moving the cart body.

The control section 81 of the control apparatus 18 registers the weight of the endoscopes mounted on the cart C (S1). For example, it may be designed such that the medical professional operates an operation section (not shown) to instruct the control section 81 to register the weight. This causes the control section 81 to receive the measurement result, i.e., the measurement result of the weight of the unused endoscopes held in the collection-requiring medical device storage section 30, from the weight measurement unit 45 via the input and output section 82. The control section 81 registers the received weight of the unused endoscopes in the memory 84. The control section 81 may be designed to receive the measurement result of the weight measurement unit 45 at a predetermined timing regardless of the user operation.

In S2, the control section 81 controls the motor control section 86 to permit the power assist of the wheels 73 by the assist motor 74. As described above, the power assist by the assist motor 74 occurs if the first propulsion force by the user can be obtained. Here, it can be assumed that a medical professional who has loaded the necessary medical devices onto the cart C moves forward and backward the cart C to transport the medical devices to the endoscope room. If the control section 81 detects that the cart C can be about to be moved forward and backward based on the detection result of the torque detection section 85, the control section 81 instructs the motor control section 86 to power assist the wheels 73. This causes the motor control section 86 to control the motor drive section 87 to drive the assist motor 74 to generate a second propulsion force, thus assisting the rotation of the wheels 73 (S3).

The medical professional transports the cart C to the endoscope room, and performs an operation to register the start of the procedure. The control section 81 registers the start of the procedure based on the operation of the medical professional (S4).

The medical professional takes out the medical device from the cart C according to the procedure. The weight measurement unit 45 measures the weight of the unused endoscopes held in the unused endoscope holding section 30A, and outputs the measurement result to the control apparatus 18 (S5). The control section 81 registers the weight of the unused endoscopes remaining in the unused endoscope holding section 30A in the memory 84 (S6). The medical professional uses the medical device taken out from the cart C for the procedure.

On finishing one procedure, the medical professional stores the medical device used in the procedure in the collection box 50. The weight measurement unit 65 (at least one weight sensor) then measures the weight of the used endoscopes stored in the used endoscope storage section 30B and outputs the measurement result to the control apparatus 18 (S7). The endoscope storage case 40 may be stored, e.g., in a collection box (not shown) separate from the collection box 50. The endoscope storage case 40 that can be relatively small may in some cases be stored in the collection box 50. The at least one weight sensor 65 can be configured to: weigh the medical devices 41 contained in the first storage area 30A to determine a first weight; and weigh the medical devices 41 contained in the second storage area 30B to determine a second weight. The at least one weight sensor 65 can be located at at least one of the first and the second storage areas (30A, 30B) of the cart body.

In S8, the control section 81 determines whether the used medical devices have been stored in the collection box 50. If the used medical devices have not been stored in the collection box 50, the process proceeds to S10. If the used medical devices have been stored in the collection box 50, the control section 81 registers the weight of the used medical devices (S9). That is, the control section 81 registers the weight of the used endoscopes based on the received measurement result.

In S10, the control section 81 determines whether or not the use of the medical device has been completed. If the use has not been completed, the control section 81 returns the process to S5. If the use has been completed, the control section 81 proceeds the process to S11, and registers an end of the procedure (S11).

Next, in S12, the control section 81 determines whether or not to permit the power assist by the assist motor 74. That is, the control section 81 subtracts from the total weight of the mounted endoscopes, can be the unused medical devices (registered in S1), the total weight of the used endoscopes, can be the used medical devices stored in the collection box 50 (registered in S9), to obtain a difference. If the calculated difference results in “all collected”, i.e., the difference being less than the weight of one endoscope, can be a medical device, the control section 81 performs control to permit the wheel power assist, and if the calculated difference results in “not all collected”, i.e., the difference being equal to or greater than the weight of one endoscope, the control section 81 performs control to not permit the wheel power assist (S12). If an unused endoscope remains in the hanger section 35 after the end of the procedure, the control section 81 may subtract from the total weight of the mounted endoscopes (registered in S1), the total weight of the used endoscopes stored in the collection box (registered in S9) and the total weight of the unused medical devices (registered in S6), to calculate the difference. If this difference can be less than the weight of one unused medical device, the control section 81 performs control to permit the assist apparatus to generate the second propulsion force. Determining whether the first storage area 30A contains the medical device 41 can be based on weighing the medical devices 41 contained in the first storage area 30A; and determining whether the second storage area 30B contains the medical device 41 can be based on weighing the medical devices 41 contained in the second storage area 30B. The at least one processor 18 can be configured to: determine a first total weight of medical devices 41, wherein the first total weight of medical devices 41 can be a sum of a weight of medical devices 41 contained in the first storage area 30A determined at a first time and a weight of medical devices 41 contained in the second storage area 30B determined at the first time, determine a second total weight of medical devices 41, wherein the second total weight of medical devices 41 can be a sum of a weight of medical devices 41 contained in the first storage area 30A determined at a second time and a weight of medical devices 41 contained in the second storage area 30B determined at the second time, determine a difference in weight between the first total weight and the second total weight, compare the difference in weight to a predetermined weight value; and when the difference in weight can be less than the predetermined weight value, control the actuator 74 to allow generation of the propulsion force. The predetermined weight value can be less than one or more of (i) a weight of a single medical device 41, (ii) a weight of a package 40 covering the medical device 41, and pre-set arbitrary weight. The second storage 30B can be configured to store the disinfectant 55, and the second total weight does not include a weight of the disinfectant 55. The at least one processor 18 can be configured to: receive a first detection result from the sensor corresponding to the first storage area 30A and the second storage area 30B containing the medical device 41, receive a second detection result from the sensor corresponding to the first storage area 30A and the second storage area 30B not containing the medical device 41, and based on the received first detection result and received second detection result, determine whether the first storage area 30A contains the medical device 41 and whether the second storage area 30B contains the medical device 41. The sensor can be one or more light sensor, image sensor, and magnetic sensor. The first and/or second detection result can include at least one of image information of the medical device, model information of the medical device, unique ID information of the medical device, number of medical devices, and type of medical device. The at least one processor 18 can be configured to: receive a first detection result from the image sensor corresponding to the first storage area 30A and the second storage area 30B containing the medical device 41, receive a second detection result from the image sensor corresponding to the first storage area 30A and the second storage area 30B not containing the medical device 41, and based on the received first detection result and received second detection result, determine whether the first storage area 30A contains the medical device 41 and whether the second storage area 30B contains the medical device 41.

In the case of “all collected,” the control section 81 controls the motor control section 86 to enable the power assist. In this case, if the medical professional applies a first propulsion force to the cart C to move the cart C for disposal of the used medical devices, the motor drive section 87 drives the assist motor 74 to apply the second propulsion force of the assist motor 74 to the wheels 73, thus performing the power assist.

In the case of “not all collected,” the control section 81 controls the motor control section 86 to not permit the power assist. In this case, even if the medical professional applies the first propulsion force to the cart C to move the cart C for disposal of the used medical devices, the assist motor 74 can be not driven and the power assist can be not performed. As a result, the medical professional needs to apply a relatively large propulsion force to the cart C to move the cart C, allowing the medical professional to recognize that not all of the used medical devices have been collected. In the case of “not all collected,” the control section 81 may output a signal to the monitor 17, 27, etc. to issue a warning by voice or an image indicating that not all of the used medical devices have been collected. This warning allows the medical professional to recognize that not all of the used medical devices have been collected.

### (When not Using Collection-Requiring Medical Device Storage Section 30)

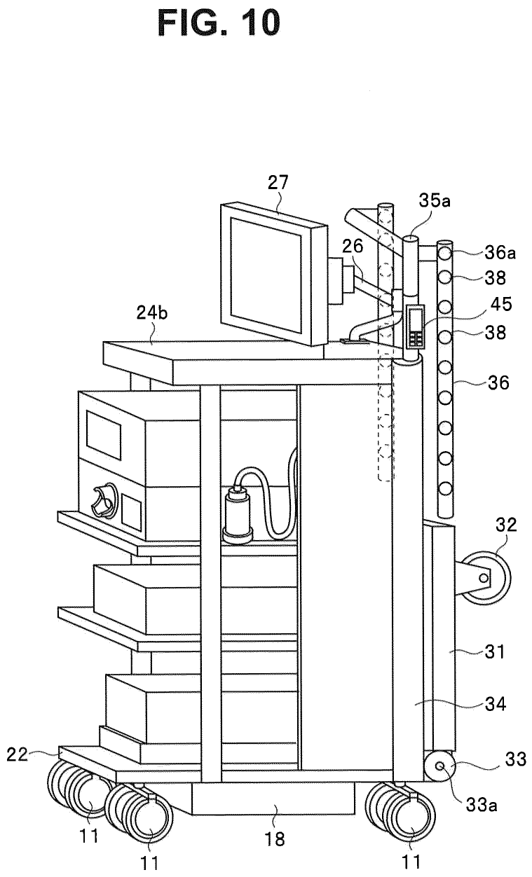

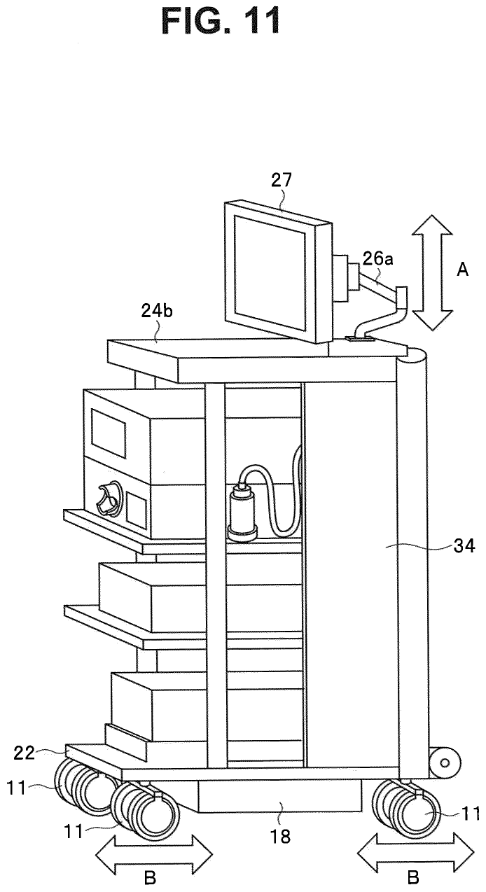

FIG. 10 is a perspective view showing an example of a state in which the collection-requiring medical device storage section 30 is not used in the cart C.

The wheeled base 31 on which to set the collection box 50 and the hanger section 35 from which a plurality of single-use endoscopes are to be hung are foldable as shown in FIG. 10. In that case, the cart can be usable as a normal endoscopic cart. In the example in FIG. 10, the wheeled base 31 can be rotated around the connecting section 33 to fold the wheeled base 31 to the rear end of the cart C1, C2. The connecting section 33 can be provided with an assist control permission switch 33a. The hanger section 35 can be configured so that the holding member 36 can be bendable on a shaft near the support pillar 35a, and this bending part can be provided the assist control permission switch 36a. Folding the hanger section 35 from the bending part enables to align most of the hanger section 35 with the rear end of the cart C1, C2. In other words, the unused endoscope holding section 30A and the used endoscope storage section 30B can be each selectively switchable between a state of protruding from and a state of being folded with respect to the endoscope apparatus storage section 10, 20.

It may be configured such that the wheeled base 31 and the hanger section 35 can be folded and unfolded to turn on and off an assist control permission switch (not shown). That is, when the wheeled base 31 and the hanger section 35 can be folded to turn off the assist control permission switch, the control section 81 determines from the state of the assist control permission switch that the collection-requiring medical device storage section 30 can be not to be used, and conducts power assist as usual. In this case, the cart C can be, e.g., always in a state in which power assist can be permitted. At least one of the first storage area 30A and the second storage area 30B can be configured to be switched between the unfolded state and the folded state. In the folded state, the at least one weight sensor may not detect the first weight and the second weight. The at least one processor can be configured to control the actuator to allow generation of the propulsion force in the unfolded state, and to prevent generation of the propulsion force in the folded state.

On the other hand, when the wheeled base 31 or the hanger section 35 can be unfolded to turn on the assist control permission switch, the control section 81 determines from the state of the assist permission switch that the collection-requiring medical device storage section 30 can be to be used, thus controlling the power assist depending on “all collected” or “not all collected”.

Not only the power assist control but the power supply for weight measurement may be turned on and off. For example, when the wheeled base 31 and the hanger section 35 can be folded to turn off the assist control permission switch, the weight measurement operation by the weight measurement units 45, 65 may be stopped (turned off). When the wheeled base 31 and the hanger section 35 can be unfolded to protrude from the endoscope storage section 10, 20, thereby turning on the assist control permission switch, the weight measurement may be turned on by the weight measurement units 45, 65.

For the sake of simplicity, it has been described assuming that the measurement result of the weight measurement unit 45 indicates the total weight of the unused medical devices held in the hanger section 35 and the measurement result of the weight measurement unit 65 indicates the total weight of the used medical devices stored in the collection box 50, and that information on these total weights can be registered in the memory 84. In practice, however, the weights of components other than the unused medical devices and the used medical devices can be also taken into consideration as described above. For example, in a case where the packaging of medical devices, such as single-use medical devices, can be disposed of at a separate location from the medical devices, the total weight of the packaging may be included in the calculation and counted along with the total weight of the collected devices. In a case of providing a packaging collection box for disposing of the packaging or disposing of the packaging at a separate location, a code indicating the weight of the packaging for each model may be read and deducted each time of use.

For example, the reading section 52 may extract information on the weight of the endoscope storage case 40 in which to package unused medical devices, and the control section 81 may perform control to permit the assist apparatus to generate the second propulsion force based on a result of subtracting the weight of the endoscope storage case 40 from the weight of the collection box 50. When measuring the weight of the collection box, the weight of the box itself and the weight of a simple disinfectant solution to be filled inside the collection box before the procedure may be pre-subtracted from the measurement.

Furthermore, the control section 81 may determine the number or type of used medical devices stored in the collection box 50 based on the image information, model information, and unique ID information of the medical devices read by the reading section 52, and perform control to permit the assist apparatus to generate the second propulsion force based on the result of the determination.

Forgetting putting the used medical devices into the collection box 50 results in the weight of the collection box 50 being lighter than expected, allowing for detection that the used medical devices have not been collected properly. Conversely, the used medical devices may become slightly heavier due to bodily fluid stuck on the devices, but this case has no problem because the difference of the result of the subtraction will change to the negative side. Accessories such as treatment tools and forceps caps may be disposed of by the hospital or may not be measured for weight, or may be stored in a collection box different from an endoscope collection box.

Thus, in this embodiment, in a case where there are not enough medical devices such as endoscopes that should be collected, the power assist can be not provided sufficiently, allowing the users such as a medical professional to notice that used single-use medical devices have not all been properly put in the collection section. This enables to promote the proper operation of single-use medical devices from the view point of safety and reuse prevention.

### (Modification)

FIG. 11 is a perspective view showing a modification. In FIG. 11, the same components as those in FIG. 2 are attached with the same reference numerals and the description can be omitted. The example in FIG. 11 configures a cart that hardly tips over by having the center of gravity that is easily changeable between when in use and when moving of the cart.

In the example in FIG. 11, a monitor arm 26a can be attached to the upper end of the top board 24b. A monitor 27 can be attached to the monitor arm 26a. The monitor arm 26a can be extendable and retractable, and by extension and retraction, the position of the monitor 27 can be changeable up and down as shown by an arrow A. For example, the bending angle of the monitor arm 26a may be configured to be changeable. For example, the monitor arm 26a may be configured with an extendable and retractable member, and locked to thereby control whether or not to allow the monitor arm 26a to extend and retract.

In the example in FIG. 11, the casters 11 can be configured so that the mounting positions on the bottom surface of the frame base 22 can be electrically or manually shifted in the longitudinal direction of the frame base 22 (the direction of arrows B in FIG. 11). That is, the casters 11 can be designed to be movable in the longitudinal direction of the frame base 22, so that the distance between the front and rear casters 11 can be changeable. This enables to increase and decrease the distance (wheel base) between the front and rear casters 11. For example, lengthening the wheel base during movement of the cart enables to ensure running stability during the movement.

Thus, in this modification, the position of the center of gravity of the cart, which tends to be high when the cart holds medical devices, can be adjustable as necessary, and the wheel base can be extendable, yielding an effect to make it difficult for the cart to tip over during movement.

The disclosure is not limited to the above-described embodiments and can be embodied by modifying the components within the scope of the gist of the disclosure at the implementation stage. Various disclosure can also be formed by appropriately combining a plurality of components disclosed in the above embodiments. For example, some of all the components shown in the embodiments may be omitted. Furthermore, components from different embodiments may be combined as appropriate.

Among the technologies described herein, the controls and functions described primarily using flowcharts can largely be configured through a program. By having a computer read and execute such a program, the aforementioned controls and functions can be realized. The program can be recorded or stored entirely or partially, as a computer program product, on portable media such as a flexible disks, CD-ROM, and non-volatile memory, and on storage media such as a hard disk and volatile memory. The program can be distributed or provided at the time of product shipment or via portable media or through a communication line. Users can download the program via a communication network and install the downloaded program on a computer, or install the program on a computer from a recording medium, to thereby easily realize the medical device transportation cart of the embodiments.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

Example 1. A medical device transportation cart comprising:
a cart body including a frame;
a holding section configured to hold an unused medical device;
a storage section configured to store a used medical device;
an assist apparatus for propelling a cart, the assist apparatus being configured to supplement a first propulsion force applied by a user and generate a second propulsion force; and
a processor, wherein
the processor is configured to:
   detect a storage state in the storage section with regard to the medical device held in the holding section; and
   perform control whether or not to permit the assist apparatus to generate the second propulsion force depending on whether or not the processor detects that the number of the used medical devices stored in the storage section is smaller than the number of the unused medical devices held by the holding section.
      Example 2. The medical device transportation cart according to Example 1, wherein
the processor is further configured to:
   perform control on the assisting device to generate the second propulsion force if the processor detects that the number of the used medical devices stored in the storage section is the same as the number of the unused medical devices held by the holding section; and
   perform control on the assisting device not to generate the second propulsion force if the processor detects that the number of the used medical devices stored in the storage section is smaller than the number of the unused medical devices held by the holding section.
      Example 3. The medical device transportation cart according to Example 2, wherein
the processor is configured to fix an operation of the assisting device if the processor detects that the number of the used medical devices stored in the storage section is smaller than the number of the unused medical devices held by the holding section.
   Example 4. The medical device transportation cart according to Example 1 further comprising:
a weight measurement unit configured to measure a weight of the holding section holding the unused medical device and a weight of the storage section storing the used medical device, wherein
the processor is configured to base a measurement result of the weight measurement unit to subtract, from a weight of the unused medical devices initially held by the holding section, a sum of a weight of the unused medical devices held by the holding section and a weight of the used medical devices stored in the storage section, to obtain a difference, and to permit the assisting device to generate the second propulsion force if the difference is less than a predetermined value.
   Example 5. The medical device transportation cart according to Example 4, wherein the processor is configured to perform control to permit the assist apparatus to generate the second propulsion force if the difference is less than a weight of one item of the unused medical device.
   Example 6. The medical device transportation cart according to Example 5, wherein
the unused medical device held in the holding section is stored in a package whose inside is kept in a sterile state, and
the processor is configured to record a weight of the package that packages the unused medical device held by the holding section, and to perform control to permit the assisting device to generate the second propulsion force based on a measurement result obtained by subtracting the weight of the package from the weight of the used medical devices stored in the storage section.
   Example 7. The medical device transportation cart according to Example 4, wherein
the storage section includes a collection box for the medical devices, the collection box having a box shape, allowing pouring-in of a disinfectant, and being liquid-tight against the outside, and
the processor is configured to record a weight of the collection box and a weight of the disinfectant poured into the collection box, and perform control to permit the assisting device to generate the second propulsion force based on a measurement result obtained by subtracting the weights of the collection box and the disinfectant from the weight of the storage section storing the used medical devices.
   Example 8. The medical device transportation cart according to Example 4, wherein
the unused medical device held in the holding section is stored in a package whose inside is kept sterile, and
the medical device transportation cart further comprises an other storage section the weight of which is not measured, the another storage section being configured to store the package from which the unused medical device has been removed.
   Example 9. The medical device transportation cart according to Example 4, wherein the weight measurement unit is placed at a part that supports the weights of the holding section and the storage section.
   Example 10. The medical device transportation cart according to Example 9, wherein the weight measurement unit that measures the weight of the storage section storing the used medical device includes a scale placed on an upper surface of a frame that supports the holding section.
   Example 11. The medical device transportation cart according to Example 4 further comprising:
a reading section configured to read information on the medical device to be stored in the storage section, wherein
the processor is configured to extract information on a weight of a package that packages the unused medical device of the information on the medical device read by the reading section, and to perform control to permit the assisting device to generate the second propulsion force based on a measurement result obtained by subtracting the weight of the package from a weight of the used medical devices stored in the storage section.
   Example 12. The medical device transportation cart according to Example 1, further comprising:
a reading section configured to read information on the medical device to be stored in the storage section, wherein
the processor is configured to perform control to permit the assisting device to generate the second propulsion force based on image information, model information, and unique ID information on the medical device read by the reading section, and based on a number or type of the medical devices stored in the storage section.
   Example 13. The medical device transportation cart according to Example 1, wherein at least one of the holding section and the storage section is configured to be selectively switchable between a state of protruding from and a state of being folded with respect to the cart body.
   Example 14. The medical device transportation cart according to Example 13, wherein
at least one of the holding section and the storage section is provided with a switch so that an operation of the weight measurement unit is turned on if the at least one is in the state of protruding from the cart body and the operation of the weight measurement unit is turned off if the at least one is in the state of being folded with respect to the cart body.
   Example 15. The medical device transportation cart according to Example 1, wherein the medical device is a single-use endoscope that is stored in the storage section after a single use and subsequently disposed of.
   Example 16. A medical device transportation cart comprising:
a cart body including a frame;
a holding section configured to hold an unused medical device;
a storage section configured to store a used medical device;
an assist apparatus for propelling a cart, the assist apparatus being configured to supplement a first propulsion force applied by a user and generate a second propulsion force; and
a processor, wherein
the processor is configured to:
   detect a storage state in the storage section with regard to the medical device held in the holding section; and
   perform control not to permit the assist apparatus to generate the second propulsion force if the processor detects that the number of the used medical devices stored in the storage section is smaller than the number of the unused medical devices held by the holding section.
      Example 17. An operating method of a medical device transportation cart, the method comprising:
measuring a weight of an unused medical device held by a holding section provided on a cart body including a frame;
measuring a weight of a used medical device stored in a storage section;
recording the weight of the unused medical device and the weight of the used medical device; and
perform control to permit an assist apparatus for propelling a cart to perform an operation to supplement a first propulsion force applied by a user to generate a second propulsion force if a difference between the weight of the used medical device stored in the storage section and the weight of the unused medical device held in the holding section is less than a predetermined value.
   Example 18. A transportation cart, comprising:
a cart body;
a first storage area configured to store a medical device before use;
a second storage area configured to store the medical device after use; and
at least one processor configured to:
   determine whether the first storage area contains the medical device;
   determine whether the second storage area contains the medical device; and
   when one of the first storage area and the second storage area is determined to contain the medical device, control an actuator to allow generation of a propulsion force for moving the cart body.
      Example 19. The transportation cart according to Example 18, wherein the at least one processor is further configured to:
when both the first and the second storage areas are determined to not contain the medical device, control the actuator to prevent generation of the propulsion force.
   Example 20. The transportation cart according to Example 18, wherein the propulsion force is a first propulsion force,
wherein the at least one processor is further configured to:
   when both the first and the second storage areas are determined to not contain the medical device, control the actuator to allow generation of a second propulsion force for moving the cart body, and
wherein the second propulsion force is less than the first propulsion force.
   Example 21. The transportation cart according to Example 18, wherein the at least one processor is further configured to:
determine a first total number of medical devices, wherein the first total number of medical devices is a sum of a number of medical devices contained in the first storage area determined at a first time and a number of medical devices contained in the second storage area determined at the first time, and
determine a second total number of medical devices, wherein the second total number of medical devices is a sum of a number of medical devices contained in the first storage area determined at a second time and a number of medical devices contained in the second storage area determined at the second time.
   Example 22. The transportation cart according to Example 21, wherein determining whether the first and the second storage areas contain the medical device comprises determine a difference in number between the first total number and the second total number, and
the at least one processor is further configured to, when the first total number and the second total number are different, control the actuator to prevent generation of the propulsion force.
   Example 23. The transportation cart according to Example 21, wherein determining whether the first and the second storage areas contain the medical device comprises determine a difference in number between the first total number and the second total number, and
the at least one processor is further configured to, when the second total number is smaller than the first total number, control the actuator to prevent generation of the propulsion force.
   Example 24. The transportation cart according to Example 18, wherein determining whether the first storage area contains the medical device is based on weighing the medical devices contained in the first storage area; and
wherein determining whether the second storage area contains the medical device is based on weighing the medical devices contained in the second storage area.
   Example 25. The transportation cart according to Example 18, wherein the at least one processor is configured to:
determine a first total weight of medical devices, wherein the first total weight of medical devices is a sum of a weight of medical devices contained in the first storage area determined at a first time and a weight of medical devices contained in the second storage area determined at the first time,
determine a second total weight of medical devices, wherein the second total weight of medical devices is a sum of a weight of medical devices contained in the first storage area determined at a second time and a weight of medical devices contained in the second storage area determined at the second time,
determine a difference in weight between the first total weight and the second total weight,
compare the difference in weight to a predetermined weight value; and when the difference in weight is less than the predetermined weight value, control the actuator to allow generation of the propulsion force.
   Example 26. The transportation cart according to Example 25, wherein the predetermined weight value is less than one or more of (i) a weight of a single medical device and (ii) a weight of a package covering the medical device.
   Example 27. The transportation cart according to Example 24, further comprising:
at least one weight sensor configured to:
   weigh the medical devices contained in the first storage area to determine a first weight; and
   weigh the medical devices contained in the second storage area to determine a second weight.
      Example 28. The transportation cart according to Example 25, wherein the second storage is configured to store a disinfectant, and
wherein the second total weight does not include a weight of the disinfectant.
   Example 29. The transportation cart according to Example 18, further comprising a light sensor located at the cart body,
wherein the at least one processor is configured to:
   receive a first detection result from the light sensor corresponding to the first storage area and the second storage area containing the medical device,
   receive a second detection result from the light sensor corresponding to the first storage area and the second storage area not containing the medical device, and
   based on the received first detection result and received second detection result, determine whether the first storage area contains the medical device and whether the second storage area contains the medical device.
      Example 30. The transportation cart according to Example 18, further comprising an image sensor located at the cart body,
wherein the at least one processor is configured to:
   receive a first detection result from the image sensor corresponding to the first storage area and the second storage area containing the medical device,
   receive a second detection result from the image sensor corresponding to the first storage area and the second storage area not containing the medical device, and
   based on the received first detection result and received second detection result, determine whether the first storage area contains the medical device and whether the second storage area contains the medical device.
      Example 31. The transportation cart according to Example 29, wherein the first detection result includes at least one of image information of the medical device, model information of the medical device, unique ID information of the medical device, number of medical devices, and type of medical device, and
wherein the second detection result includes at least one of image information of the medical device, model information of the medical device, unique ID information of the medical device, number of the medical devices, and type of the medical devices.
   Example 32. The transportation cart according to Example 18, wherein at least one of the first storage area and the second storage area is configured to be switched between an unfolded state and a folded state.
   Example 33. The transportation cart according to Example 18, wherein the medical device is a single-use endoscope.
   Example 34. The transportation cart according to Example 18, wherein the propulsion force is a first propulsion force, and
wherein the at least one processor is configured to:
   receive a signal from a detector of application to the cart body of a second propulsion force for moving the cart body, and
   in response to receiving the signal, control the actuator to generate the first propulsion force.
      Example 35. An operating method of a transportation cart, the method comprising:
determining whether a first storage area of the transportation cart contains a medical device;
determining whether a second storage area of the transportation cart contains the medical device; and
when one of the first storage area and the second storage area is determined to contain the medical device, control an actuator to allow generation of a propulsion force for moving the cart body.
   Example 36. A transportation cart, comprising:
a cart body;
a storage area configured to store a medical device;
an actuator configured to generate a propulsion force for moving the cart body; and
at least one processor configured to:
   determine a first number of the medical device contained in the storage area at a first time,
   determine a second number of the medical device contained in the storage area at a second time, and
   when the first number and the second number are different, control the actuator to prevent generation of the propulsion force.
      Example 37. The transportation cart according to Example 36, wherein the at least one processor is further configured to, when the first number and the second number are the same, control the actuator to allow generation of the propulsion force.

What is claimed is:

1. A transportation cart, comprising:
a cart body;
a storage area configured to store a medical device; and
at least one processor configured to:
determine a first number of the medical device contained in the storage area at a first time,
determine a second number of the medical device contained in the storage area at a second time, and
when the first number and the second number are different, control an actuator to restrict generation of the propulsion force.

2. The transportation cart according to claim 1, wherein the at least one processor is further configured to:
when the first number and the second number are the same, control the actuator to allow generation of the propulsion force.

3. The transportation cart according to claim 1, wherein the storage area comprises:
a first storage area configured to contain the medical device before use; and
a second storage area configured to store the medical device after use,
wherein the first number of the medical device is a number of the medical device contained the first and the second storage areas at the first time, and
wherein the second number of the medical device is a number of the medical device contained the first and the second storage areas at the second time.

4. The transportation cart according to claim 1, wherein the at least one processor is further configured to:
when the second number is smaller than the first number, control the actuator to restrict generation of the propulsion force.

5. The transportation cart according to claim 1, wherein the at least one processor is further configured to:
when the first number and the second number are different, prevent the actuator not to generate the propulsion force.

6. The transportation cart according to claim 4, wherein the at least one processor is further configured to:
when the second number is smaller than the first number, control the actuator not to generate the propulsion force.

7. The transportation cart according to claim 3, wherein the at least one processor is configured to:
determine a first total weight of medical devices, wherein the first total weight of medical devices is a sum of a weight of medical devices contained in the first storage area determined at the first time and a weight of medical devices contained in the second storage area determined at the first time,
determine a second total weight of medical devices, wherein the second total weight of medical devices is a sum of a weight of medical devices contained in the first storage area determined at the second time and a weight of medical devices contained in the second storage area determined at the second time,
determine a difference in weight between the first total weight and the second total weight,
compare the difference in weight to a predetermined weight value, and
when the difference in weight is less than the predetermined weight value, control the actuator to allow generation of the propulsion force.

8. The transportation cart according to claim 3, wherein determining whether the first storage area contains the medical device is based on weighing the medical devices contained in the first storage area, and
wherein determining whether the second storage area contains the medical device is based on weighing the medical devices contained in the second storage area.

9. The transportation cart according to claim 8, further comprising:
at least one weight sensor configured to:
weigh the medical devices contained in the first storage area to determine a first weight, and
weigh the medical devices contained in the second storage area to determine a second weight.

10. The transportation cart according to claim 7, wherein the predetermined weight value is less than one or more of (i) a weight of a single medical device and (ii) a weight of a package covering the medical device before use in a sterile state.

11. The transportation cart according to claim 8, wherein the second storage is configured to store a disinfectant, and
wherein the second total weight does not include a weight of the disinfectant.

12. The transportation cart according to claim 8, wherein the at least one weight sensor is located at at least one of the first and the second storage areas of the cart body.

13. The transportation cart according to claim 3, further comprising a sensor located at the cart body,
wherein the at least one processor is configured to:
receive a first detection result from the sensor corresponding to the first storage area and the second storage area containing the medical device,
receive a second detection result from the sensor corresponding to the first storage area and the second storage area not containing the medical device, and
based on the received first detection result and received second detection result, determine whether the first storage area contains the medical device and whether the second storage area contains the medical device.

14. The transportation cart according to claim 3, further comprising an image sensor located at the cart body,
wherein the at least one processor is configured to:
receive a first detection result from the image sensor corresponding to the first storage area and the second storage area containing the medical device,
receive a second detection result from the image sensor corresponding to the first storage area and the second storage area not containing the medical device, and
based on the received first detection result and received second detection result, determine whether the first storage area contains the medical device and whether the second storage area contains the medical device.

15. The transportation cart according to claim 13, wherein the first detection result includes at least one of image information of the medical device, model information of the medical device, unique ID information of the medical device, number of medical devices, and type of medical device, and
wherein the second detection result includes at least one of image information of the medical device, model information of the medical device, unique ID information of the medical device, number of the medical devices, and type of the medical devices.

16. The transportation cart according to claim 7, wherein at least one of the first storage area and the second storage area is configured to be switched between an unfolded state and a folded state, and
wherein in the folded state, the at least one weight sensor doesn't detect the first weight and the second weight.

17. The transportation cart according to claim 1, wherein the medical device is a single-use endoscope.

18. The transportation cart according to claim 1, wherein the propulsion force is a first propulsion force, and
wherein the at least one processor is configured to:
receive a signal from a detector of application to the cart body of a second propulsion force for moving the cart body, the second propulsion force is applied from a medical person, and
in response to receiving the signal, control the actuator to generate the first propulsion force.

19. A transportation cart according to claim 1, further comprising:
the actuator configured to generate the propulsion force for moving the cart body.

20. An operating method of a transportation cart, the method comprising:
determining a first number of a medical device contained in the storage area at a first time;
determining a second number of the medical device contained in the storage area at a second time; and
when the first number and the second number are different, control an actuator to restrict generation of a propulsion force for moving the cart body.
